(19) United States
(12) Patent Application Publication
(10) Pub. No.: US 2018/0338929 A1
(43) Pub. Date: Nov. 29, 2018

(54) Method and Compounds for Treating Peripheral Neuropathy

(71) Applicant: Soon Kap Hahn, Irvine, CA (US)

(72) Inventor: Soon Kap Hahn, Irvine, CA (US)

(21) Appl. No.: 15/986,533

(22) Filed: May 22, 2018

Related U.S. Application Data
(60) Provisional application No. 62/510,240, filed on May 23, 2017.

(51) Int. Cl. A61K 9/50; A61K 31/165; A61K 31/192

(52) U.S. Cl. CPC A61K 9/5031; A61K 31/192; A61K 31/165

(57) ABSTRACT

The present disclosure relates to a to a method for treating peripheral neuropathy including diabetic neuropathy. It consists of comprehensive set of pain management medications such as sodium channel blockers, N-methyl-D-aspartate (NMDA) receptor antiagonists, α-2 adrenergic receptor agonists, anti-inflammatory drugs, calcium channel blockers, other classes of medications or their mixtures. The medical compound can be encapsulated in PLGA micro-particles for controlled, sustained release over 2-8 weeks. Once a doctor prescribes a set of pain management medications, the prescribed medication(s) can be administered individually or as their mixtures by a commercial painless or user-friendly micro-needle patch or injection device.

### CROSS-REFERENCE TO RELATED APPLICATIONS

This application claims the benefit of U.S. Provisional Application 62/510,240 filed on May 23, 2017.

### FIELD OF THE INVENTION

The present invention relates generally to method for treating peripheral neuropathy including diabetic neuropathy. More specifically, this invention relates to a comprehensive set of pain management medications such as sodium channel blockers, N-methyl-D-aspartate (NMDA) receptor antiagonists, α-2 adrenergic receptor agonists, anti-inflammatory drugs, calcium channel blockers, other classes of medications or their mixtures.

### BACKGROUND OF THE INVENTION

Peripheral neuropathy, a result damage to your peripheral nerves, often causes weakness, numbness and pain, usually affects the hands and feet but it can also affect other areas of the human body. An estimated 2 million people in the United States have some form of peripheral neuropathy.

Diabetic neuropathy is one of the most common forms of peripheral neuropathy and a type of nerve damage that can occur in diabetes (type 1 or 2). High blood glucose level due to diabetes can damage nerve fibers throughout body, but diabetic neuropathy most often damages nerves in hands and feet. It causes symptoms such as tingling, numbness, burning and pain. These symptoms are mild for some people, but they can be painful, disabling and even fatal for others. The pathology of diabetic neuropathy is still unknown and there is no treatment to cure diabetic neuropathy. Current treatment is to reduce the pain associated with diabetic neuropathy (symptomatic treatment).

The symptomatic treatment typically involves the use of antidepressants, anticonvulsants or opioid or opioid-like medications taken orally. Concerns related to potential side effects with these oral medications prevent their use in many patients. Most of these drugs require systemic effects mainly on spinal cord and/or brain (i.e. central nervous system) to reduce the pain caused by diabetic neuropathy. However, antidepressants and anticonvulsants taken orally Possess significant side effects such as insomnia, dizziness, dry mouth, weight gain, headache and nausea. The long-term use of opioids or opioid-like medications may cause addiction. So far, there are only three oral medications approved by the FDA for treating diabetic neuropathy; duloxetine (“Cymbalta”, anti-depressant), pregabalin (“Lyrica”, anti-convulsant) and tapentadol (“Nucynta”, opioid). They are known to be suboptimal in reducing pains with only about 50% effective for diabetic neuropathy patients.

Numerous mechanisms related to transduction or transmission functions have been linked in causing pain. These transduction or transmission functions involve multiple receptors. Typically, the above oral medications for treating painful diabetic neuropathy act at one specific receptor site. Each patient may have different mechanism(s) for causing their pain. This may explain why, for example, antidepressants show a good efficacy for some patients but not for other patients. Since it is difficult to predict in each patient to target a correct receptor site, the outcome of prescribed medications is unpredictable. Using multiple oral medications for targeting various receptor sites is not a viable option due to their cumulative side effects and adverse drug-drug interactions.

Local delivery of pain management medications may reduce the pain caused by diabetic neuropathy without the systemic side effects associated with medications delivered orally. In addition, multiple medications can be administered locally to target multiple receptors without causing side effects and adverse drug-drug interactions. Since the plasma concentration of locally delivered medications is only 5 to 15 percent of the corresponding oral medications, the incidence of systemic side effects and adverse drug-drug interactions is dramatically reduced compared to the systemic use of the same medications delivered orally. There have been two methods being developed as the local delivery of pain management medications: 1) controlled, sustained delivery of pain medications encapsulated in biodegradable polymer such as polylatic glycolic acid (PLGA) and 2) passive transdermal delivery of compounded pain management medications.

The pain management medications can be formulated into a biodegradable polymer such as PLGA which degrades over weeks or months. D. S. Kohane et al. taught the use of naturally occurring site 1 sodium channel blockers such as tetrodotoxin (a biological toxin) with other drugs to prolong nerve blocking duration of the biological toxin and improve safety and efficacy (U.S. Pat. No. 6,326,020).

Other drugs included a local, anesthetic, vaso-constrictor, glucocorticoid, and/or adrenergic drugs like alpha-1 agonists (phenylephrine), beta-blockers (propranolol), and alpha-2 agonists (clonidine). Their main goal of adding the other drugs was to prolong the nerve blocking duration of the biological toxin. For example, they claimed that the addition of vasoconstrictor caused a slower systemic absorption of the biological toxin and prolonged the nerve blocking duration. They also prepared PLGA microspheres of these drugs to prolong the nerve blocking duration further. W. F. McKay taught the use of opioid analgesic drug such as morphine and anti-inflammatory drug such as dexamethasone to treat inflammation and pain. These drugs were encapsulated in biodegradable polymer like PLGA to form a depot for long-term effect (U.S. Pat. No. 8,470,360; U.S. Pat. No. 9,265,733; U.S. Pat. No. 9,301,946). The same author also taught the use of clonidine and GABA compound to treat inflammation and pain using PLGA microsphere formulations (U.S. Pat. No. 9,301,946). N. Bodick et al. taught the use of corticosteroid such as triamcinolone acetonide (TCA) encapsulated in PLGA microspheres for treating joint pain caused by osteoarthritis or rheumatoid arthritis (U.S. Pat. Nos. 8,828,440; 9,555,047; 9,555,048). J. M. Criscione et al. taught the use of anticonvulsant such as carbamazepine encapsulated in PLGA microspheres to treat acute, chronic or post-operative pain (US publication #20160317446; #20160136094; #20160136179). R. Ohri et al. taught the use of local anesthesic drugs such as lidocaine encapsulated in PLGA microspheres to treat, chronic pain at least 28 days (US publication #20160089335). M. Chasin et al. taught the use of bupivacaine encapsulated in PLGA microspheres to induce local analgesia, local anesthesia or nerve blockage for at least one day MS publication #20030152637). W. M. Vaughn et al. taught the use of non-steroidal anti-inflammatory drugs and lidocaine, a local analgesic drug, encapsulated in PLGA (U.S. Pat. No. 6,528,097). C. B. Berde et al. taught the use of local anesthetic drug and glucocorticosteroid encapsulated in PLGA to prolong local numbness or pain relief (U.S. Pat. No.5,700,485; U.S. Pat. No. 5,922,340).

Jacobs also taught the use of passive transdermal delivery system of compounded pain management medications in a cream formulation. This method included a comprehensive set of pain management medications including anti-inflammatory, local anesthetic, calcium channel blocker, gabapentin, tricyclic anti-depressant, baclofen, clonidine, ketamine and other drugs (Podiatry Today, vol 28, issue 3, March 2015).

The above prior arts taught the use of various pain management medications encapsulated in PLGA microspheres for treating acute or chronic pains including neuropathic pains. However, none of these methods include comprehensive set of pain management medications. The pathology of pain, especially neuropathic pain including diabetic neuropathy, is not well elucidated and may involve multiple receptors. Effective treatment should consider inclusion of a comprehensive set of pain management medications. The passive transdermal delivery of compounded pain management medications may provide delivery of comprehensive pain management medications without causing systemic side effects However, the passive diffusion of pain management medications across the skin is inconsistent in delivery amount. In addition, this method requires topical application several times per day, which is cumbersome, Therefore, there is a need to develop more effective, convenient method for treating diabetic neuropathy.

### SUMMARY OF THE INVENTION

The present invention relates to a method for treating peripheral neuropathy including diabetic neuropathy. It consists of comprehensive set of pain management medications such as sodium channel blockers, N-methyl-D-aspartate (NMDA) receptor antiagonists, α-2 adrenergic receptor agonists, anti-inflammatory drugs, calcium channel blockers, other classes of medications or their mixtures. It provides regulations of multiple receptors simultaneously without significant side effects, In addition, it provides a treatment method which can be tailored to a prescription determined by doctor for each patient. For example some patient may not need specific medication(s) which can be easily removed from the above comprehensive set. In the present invention, these medications can be encapsulated in PLGA micro-particles for controlled, sustained release over 2-8 weeks. Once a doctor prescribes a set of pain management medications, the prescribed medication(s) can be administered individually or as their mixtures by a commercial painless micro-needle patch or injection device. This user-friendly injection method enables injecting a small amount of pain management medications at multiple sites which can enhance efficacy of treatment.

### DESCRIPTION OF THE PREFERRED EMBODIMENTS

Local controlled, sustained drug delivery system (DDS) using biodegradable polymers has been actively developed since sutures made of biodegradable polymers were successfully commercialized about 40 years ago. Among all the biodegradable polymers, polylactic glycolic acid (PLGA) has shown the most potential as, a drug delivery system due to its long clinical history and versatile degradation property. A number of drug delivery systems based on PLGA have been already commercialized. These products include Lupron Depot®, Risperdal Consta®, Zoladex Depot®, Decapetyl and Sandostatin LAR®. Their combined worldwide sales revenue is approximately $5 billion. There are many additional PLGA-based drug delivery system products under development. Drug release rate from PLGA micro-particles can be controlled by adjusting a number of parameters such as 1) ratio between polylactic acid (PLA) and polyglycolic acid (PGA), 2) molecular weight and 3) size of micro-particle. In PLGA, polylactic acid is more hydrophobic compared to polyglycolic acid and subsequently hydrolyzes (i.e. degrades) slower. For example, PLGA 50:50 (PLA:PGA) exhibits a faster degradation than PLGA 75:25 due to preferential degradation of glycolic acid proportion if two polymers have the same molecular weights. PLGA with higher molecular weight exhibits a slower degradation rate than PLGA with lower molecular weight. Molecular weight has a direct relationship with the polymer chain size. Higher molecular weight PLGA has longer polymer chain and requires more time to degrade than lower molecular weight PLGA. In addition, an increase in molecular weight decreases drug diffusion rate and therefore drug release rate. The size of micro-particle also affects the rate of drug release. As the size of micro-particle decreases, the ratio of surface area to volume of the micro-particle increases Thus, for a given rate of drug diffusion, the rate of drug release from the micro-particle will increase with decreasing micro-particle size. In addition, water penetration into smaller micro-particle may be quicker due to the shorter distance from the surface to the center of the micro-particle. In addition, the property and amount of medication can also affect the rate of drug release.

Medications

The present invention uses medication(s) having effects on various receptor sites such as sodium channel blockers, N-methyl-D-aspartate (NMDA) receptor antiagonists, α-2 adrenergic receptor agonists, anti-inflammatory drugs, calcium channel blockers, other classes of medications or their mixtures to reduce diabetic neuropathy pain, other neuropathic pains or other chronic pains such as back pains and joint pains including osteoarthritis.

Sodium Channel Blocker

Sodium channels control a flow of sodium ions that can trigger excitability of pain-sensing sensories in the peripheral nervous system. Blocking the flow of sodium ions reduces pain. In addition to reducing the pain, sodium channel blockers are useful for treating a variety of other diseases described below:
Tricyclic anti-depressants (TCAs)
Anti-convulsants
Antiarrhythmics
Local anesthetics

The present invention can select one or more sodium channel blockers described in the following sections as pain management medication(s).

TCAs

TCAs are a popular treatment choice for patients with depression. They include amitriptyline, nortriptyline, desipramine, doxepin and imipramine. The TCAs have multiple modes of action such as inhibition of serotonin and norepinephrine reuptake from synaptic clefts, varying degrees of anticholinergic receptor inhibition and blocking sodium and calcium channels. In some embodiments, the present invention uses amitriptyline as its pain management medication. Amitriptyline demonstrated strong efficacy in reducing diabetic neuropathy pain (NNT; number of patient needed to treat for at least 50% pain relief=1.3) when taken orally (S. Javed et al. Therapeutic Advances in Chronic Disease, vol 6, pp 15-28, 2015) However, it has many systemic side effects prohibiting a broad commercial use. These side effects caused by oral administration can be reduced by our local delivery system.

Anti-Convulsants

Anti-convulsants treat epileptic seizures and include a diverse group of medications such as barbiturates (phenobarbital), benzodiazepines (diazepam and lorazepam), carboxamides (carbamazepine and oxcarbazepine), fructose derivatives (topiramate), GABA analogs (pregabalin and gabapentin), hydantoins (phenytoin), sulfonamides (methazolamide) and functionalized amino acids (lacosamide). The anti-convulsants block mainly sodium channel and calcium channel and enhance GABA functions. Among them, carbamazepine, oxacarbazepine, phenytoin and lacosamide are known to be potent sodium channel blockers and can be used in our Present invention. In some embodiments, lacosamide which is more selective sodium channel blocker toward a small fiber neuropathy can be used.

Antiarrhythmics

Antiarrhythmics are a group of drugs that are used to suppress abnormal rhythms of the heart such as atrial fibrillation, atrial flutter, ventricular tachycardia, and ventricular fibrillation. Class I antiarrhythmics function as a sodium channel blocker and have three groups in Ia, Ib and Ic. Group Ia lengthens the action potential, Ib shortens the action potential and Ic does not significantly affect the action potential. Group Ia includes quinidine, procainamide and disopyramide. Group Ib includes mexiletine, lidocaine, tocainide and phenytoin. Group Ic includes flecainide, procainamide, moricizine and propafenone. In some embodiments, our invention uses quinidine, procainamide, disopyramide, mexiletine, lidocaine, tocainide, phenytoin, flecainide, procainamide, moricizine or propafenone.

Local Anesthetics

Local anesthetics are a medication used to decrease pain or sense in a specific area. They are used by injecting them into the area around a nerve. Local anesthetics based on blocking sodium channel include lidocaine, tetracaine, bupivacaine and ropivacaine. In some embodiment, our invention uses lidocaine, tetracaine, bupivacaine or ropivacaine.

In addition to the sodium channel blockers described above, the present invention can also use other sodium channel blockers such sumatriptan (migraine treatment) or rufinamide (anti-convulsant).

NMDA Receptor Antiagonists

It is known that NMDA receptor antiagonists are effective in treating neuropathic pain. NMDA receptor antiagonists include ketamine. In some embodiments, the present invention includes ketamine.

α2-Adrenergic Receptor Agonist

α2-adrenergic receptor agonists have been used for decades to treat common medical conditions such as hypertension, attention deficit hyperactivity disorder, various pain and panic disorders, symptoms of opioid, benzodiazepine/alcohol withdrawal and cigarette craving. However, in recent years, these drugs have been used for muscle relaxant, sedation and analgesia. The α2-adrenergic receptor agonists include clonidine, tizanidine and dexmedetomidine. In some embodiments, the present invention uses clonidine, tizanidine or dexmedetomidine.

Anti-Inflammatory Drugs

A variety of anti-inflammatory drugs are in use routinely for musculoskeletal pain, They reduce the pain by inhibiting prostaglandins, which lower the threshold for pain conduction and act synergistically with other agents that initiate pain, such as bradykinin, serotonin or 5-hydroxytriptamine. Anti-inflammatory drugs include ibuprofen, flurbiprofen, ketoprofen and diclofenac. In some embodiments, our invention uses ibuprofen, flurbiprofen, ketoprofen or diclofenac.

Calcium Channel Blockers

Calcium channel blockers are vasodilators and may increase neural vascular perfusion, contributing to improving any ischemic neuropathy component. Calcium channel blockers include nifedipine and verapamil. In some embodiments, our invention uses nifedipine or verapamil.

Other Medication Classes

The present invention can also use other classes of medications such as GABA analogs (gabapentin or pregabalin serotonin norepinephrine reuptake inhibitors (duloxetine, venlafaxine or desvenlafaxine), selective serotonin reuptake inhibitors (sertraline, fluoxetine, escitalopram or paroxetine) or muscle relaxant (baclofen or cyclobenzaprine). These classes of medications can be encapsulated into PLGA microparticles and administered individually or a mixture with other medications described above

Miro-Particles

Micro-particles represent an attractive means to achieve the desired local delivery of pain management medications. Micro-particles used herein refer to particles having sizes between 1 μm and 250 μm, preferably less than 50 μm and include microcapsules, microspheres and other particles. Micro-particles composed of drugs or medicaments and polymers are commonly used as a sustained, controlled release drug delivery system. Microcapsules generally have a drug core coated with a polymer film and may be spherical or non-spherical in shape. In contrast microspheres have drugs dispersed evenly in polymer and are spherical in shape.

In some embodiments, a medication having effect on a specific receptor site can be encapsulated in PLGA micro-particles individually (“PLGA formulation”). For use in patients, individual PLGA formulation can be administered alone or a mixture with other PLGA formulation(s). For example, lacosamide, a sodium channel blocker, can be encapsulated in PLGA micro-particles (“lacosamide-PLGA formulation”). Ibuprofen, an anti-inflammatory drug, can be encapsulated in PLGA micro-particles (“ibuprofen-PLGA formulation”). Depending on prescription determined by doctor patient can be treated with lacosamide-PLGA formulation, ibuprofen-PLGA formulation or a mixture of both PLGA formulations. In some embodiment, each of sodium channel blocker, NMDA receptor antagonist, α2-adrenergic receptor agonist, anti-inflammatory drug, calcium channel blocker or other classes of medications can be encapsulated in PLGA micro-particles. These PLGA formulations can be used individually or as their mixtures depending on prescription by doctor.

The composition of PLGA consists of equal to or more than 50% of polylactic acid (PLA). In some embodiment, each PLGA micro-particle contains 1-50% of medication by weight. Drug release rate from each PLGA micro-particles can be controlled by adjusting a number of parameters such as 1) ratio between polylactic acid (PLA) and polyglycolic acid (PGA), 2) molecular weight, 3) size of micro-particle and 4) amount of encapsulated medication. The present invention prepares each PLGA microsphere with a different medication having a similar drug release rate. Ideally all PLGA, microspheres with various medications release their encapsulated medications over the same period between one and two months. To adjust their drug release rates, some PLGA microsphere may contain excipients such as polyethylene glycol (PEG) or polyvinylpyrrolidone (PVP) which can accelerate the biodegradation of micro-particles. Molecular weight of PLGA is between 10,000 and 150,000 Daltons, preferably 25,000 to 75,000 Daltons.

Micro-Particle Fabrication

Micro-particles in the present invention can be prepared by microencapsulation, spray drying, precipitation, hot melt microencapsulation, co-extrusion, precision particle fabrication (PPP) or other fabrication techniques. Microencapsulation techniques use single, double or multiple emulsion process in combination with solvent removal step such, as evaporation, extraction or coacervation step, They are the most commonly used techniques to prepare micro-particles. The above techniques including the microencapsulation techniques can be used for water soluble drug, organic solvent soluble drug and solid powder drug. Polyesters can be processed with any one of the above techniques.

Excipients

Micro-particles in the present invention may also contain one or more pharmaceutically acceptable additives. The term “additive” is all components contained in micro-particles other than drugs or polymer and includes, but not limited to, buffers, preservatives and antimicrobials. It can also include hydrophilic materials such as polyethylene glycol (PEG) or polyvinylpyrrolidone (PVP) which can accelerate the biodegradation of micro-particles.

Painless Microinjection Device

Conventional hypodermic needles are often used in clinical practice to deliver medications across the skin into the bloodstream. Injections with hypodermic needles are important from a clinical standpoint, but painful. The present invention may require injections at multiple sites. Painless micro-needle injection device such as ClickSoft™ Microinjection Device by PKA SoftTouch Corp. and patch such as Microneedle Drug Delivery System by 3M have been developed and commercialized. The present invention can use one of these new injection methods to inject at single site, or multiple sites.

Treatment

A physician compounds a comprehensive set of pain management medications such as sodium channel blockers, N-methyl-D-aspartate (NMDA) receptor antiagonists, α-2 adrenergic receptor agonists, anti-inflammatory drugs, calcium channel blockers, other classes of medications or their mixtures for a specific patient. It provides regulations of multiple receptors simultaneously without significant side effects. The specific medicament compound can be supplied to the physician or independent medicament compounds can be supplied that are designed to be combined in a specific ratio for each patient treatment. In this way, it provides a treatment method which can be tailored to a prescription determined by doctor for each patient. The physician can also include a proportion of non-medications such as buffers, preservatives and antimicrobials. The medicament compound and proportion of non-medicament composite are all encapsulated in PLGA micro-particles for controlled, sustained release over 2-8 weeks. Once a doctor prescribes a set of pain management medications, the prescribed medication(s) can be administered individually or as their mixtures by a commercial painless micro-needle patch or injection device. This user-friendly injection method enables injecting a small amount of pain management medications at multiple sites which can enhance efficacy of treatment.

1. A medicament compound comprising pain management medications such as sodium channel blockers, aspartate (NMDA) receptor antiagonists, α-2 adrenergic receptor agonists, anti-inflammatory drugs, calcium channel blockers, other classes of medications or their mixtures.

2. The medicament co compound as recited in claim 1, wherein said compound in encircled by, and encapsulated within, a plurality of micro-particles.

3. The medicament compound as recited in claim 2, wherein said medicament compound encircled by, and encapsulated within, a plurality of micro-particles is injected to one or more treatment areas using a user-friendly and/or painless microinjection device.

4. The medicament compound as recited in claim 2, wherein said plurality of PLGA micro-particles.

5. The compound as recited in claim 3, wherein injected micro-particles contain a proportion of pain management medications and a proportion of buffers, preservatives and antimicrobials.

6. The medicament compound recited in claim 1 is designed to reduce diabetic neuropathy pain, other neuropathic pains or other chronic pains such as back pains and joint pains including osteoarthritis.

7. The medicament compound as recited in claim 2, wherein said compound is encircled by, and encapsulated within, a plurality of micro-particles that designed to have a controlled, sustained release of the medicaments over a period of 2-8 weeks.
